(19) United States
(12) Patent Application Publication
(10) Pub. No.: US 2005/0244809 A1
(43) Pub. Date: Nov. 3, 2005

(54) Array biosensor and method of using same for detecting the concentration of one or more analytes in one or more biological samples

(75) Inventors: Hsiao-Chung Tsai, HsinChu Hsien (TW); Ruey-An Doong, Hsinchu (TW)

Correspondence Address: BRUCE H. TROXELL, SUITE 1404, 5205 LEESBURG PIKE, FALLS CHURCH, VA, 22041, US

(73) Assignee: Zenith Cio-Tech Co., Ltd.

(21) Appl. No.: 10/836,271

(22) Filed: May 3, 2004

(51) Int. Cl. C12Q 1/00; C12Q 1/68

(52) U.S. Cl. 435/4

(57) ABSTRACT

A device for detections of one or more analytes in one or more samples is disclosed. The device can simultaneously detect multiple analytes in a sample or an analyte in a plurality of samples. The device has a plurality of wells formed thereon. Each of the well has a respective composition loaded therein, wherein each of the composition comprising a respective catalyst is encapsulated in sol-gel. In addition, a first fluorescent dye and a second fluorescent dye are encapsulated in the sol-gel or added to the sample(s) for detection and quantitation. The catalyst(s) interacts with or reacts with the specific analyte(s) in the sample(s) and causes a change(s) in spectroscopic property. The concentration of an analyte(s) is detected by comparing the normalized spectroscopic property to a standard curve. A method is also disclosed for the detection of one or more analytes in one or more samples by using the device.

### TECHNICAL FIELD OF THE INVENTION

The present invention relates to the field of detecting the concentration of one or more analytes in one or more samples by using an array biosensor. More particularly, the present invention provides a detecting device with a plurality of wells formed thereon, wherein each of the well comprises a respective catalyst encapsulated in sol-gel to react or interact with a specific analyte in the sample(s). The present invention also provides a method for detecting the concentration of one or more analytes in one or more samples simultaneous by using the detecting device.

### BACKGROUND OF THE INVENTION

A biosensor can be defined as a device intimately incorporating a biological sensing element to a transducer. Since it provides advantages of sensitivity, portability, reliability, and feasibility, the biosensor is among the fastest growing of analytical techniques for the determination of various target molecules in biomedicine, environment, chemical warfare, and food industry.

Biosensors are originally designed for a single analyte determination due to the continuous monitoring capability it behaves. However, an increasing of interested analytes and the requirement of on-site determination encourage the progress on the design and fabrication of an array-based biosensor for the accomplishment of aspirations of highly parallel and simultaneous multi-analyte analyses.

To develop an array-based biosensor comprising of different biomolecules for a rapid determination of multi-analyte, a simple, friendly and flexible immobilization method is definitely required. The immobilization techniques can be simply categorized into the non-covalent and covalent methods, or, in detail, explained by five principle strategies: physical adsorption, covalent binding, entrapment, encapsulation, and cross-linking.

Among these approaches, the covalent binding and cross-linking techniques are mainly based on the covalent attachment of biomolecules to water-insoluble matrices. It is the most wide-spread and one of the most thoroughly investigated methods for protein (enzyme) immobilization. However, complicated procedures as well as the protein denaturation are major drawbacks that should be encountered.

Sol-gel techniques provide a three-dimensional network for protein encapsulation through a simple and low temperature process. Therefore, it not only offers larger capacity for biomolecule entrapments but also preserves relatively high activity and folded conformation of proteins in comparing to covalent binding techniques. Furthermore, silica matrix is an inner and optically transparent material, making it an ideal platform for the manufacturing of optical-based biosensors.

Protein microarrays and biosensor arrays have received considerable attentions since it provides the opportunity for high-throughput analysis of protein function, screening of molecular interaction and simultaneous multi-analyte detection. A number of approaches for the manufacturing of biochips and biosensors have been developed such as screen-printing, ink-jet printing, photolithography, photopolymerization and direct deposition.

Of various techniques developed, the contact printing utilizing the robotic system with metallic pins to deliver biomolecules on solid support shows reliable characteristics and capability for high-speed array biosensor fabrication. A spot size of 100 to 500 μm with printing speed of 1 spot/sec could be generally achieved.

The immobilization method is an important parameter for array biosensor fabrication because it governs the stability and applicability of the developing system. Sol-gel technique provides an alternative to encapsulate biomolecules in porous silica, which can preserve the catalytic activity of enzymes under suitable conditions.

Sol-gel-derived biosensors have been proven to be remarkable techniques for the detection of substrates, inhibitors, cofactors, and effectors of enzymes, antigens and haptens that bind to antibodies. It is beneficial to fabricate a sol-gel-derived array biosensor comprising of different catalysts for simultaneously detecting and determining the concentration of one or more analytes in one or more human biological samples that are indicative of health.

### SUMMARY OF THE INVENTION

The present invention provides a sol-gel-derived array biosensor that is simple, easy to make for detecting the concentration of one or more analytes in one or more human biological samples simultaneously. The array biosensor has a plurality of wells formed thereon, wherein each of the well comprises a respective catalyst such as a respective enzyme encapsulated in the sol-gel. In one embodiment of the present invention, a first fluorescent dye and a second fluorescent dye are also encapsulated in the sol-gel for purposes of detection, calibration and quantitation. In another embodiment, the first fluorescent dye and the second fluorescent dye are added to one or more sample solutions comprising a respective human biological sample.

Thus, an object of the present invention is to provide a sol-gel-derived array biosensor that is simple, sensitive, accurate and convenient for detecting the concentration of one or more analytes in one or more biological samples.

Another object of the present invention is to provide a sol-gel-derived array biosensor for detecting the concentration of one or more analytes in one or more samples simultaneously.

Another object of the present invention is to provide a method for detecting the concentration of one or more analytes in one or more samples simultaneously.

Yet another object of the present invention is to provide a method of making a sol-gel-derived array biosensor for detecting the concentration of one or more analytes in one or more samples simultaneously.

In the absence of the analyte, the array biosensor displays certain baseline spectroscopic properties. However, when the analyte is present in the biological sample, changes in the spectroscopic properties are represented as the ratio of fluorescent intensity of the first fluorescent dye and the second fluorescent dye. Therefore, the concentration of the analyte(s) in the biological sample(s) is obtained basing on a comparison of the normalized property to a standard curve made by methods well known to those skilled in the art of analytical chemistry.

The present invention also includes a method of making the detecting device. In the method of making the device according to the present invention, micro-wells are formed on the upper surface of a solid support and one or more compositions comprising a respective catalyst are encapsulated in sol-gel within the micro-wells.

### BRIEF DESCRIPTION OF THE DRAWINGS

FIG. 1 shows the developed total immersion system according to the present invention.

FIG. 2 shows the response curve of urease-based array biosensor for the determination of urea according to the present invention.

FIG. 3 shows the response curve of CD-based array biosensor on the creatinine determination according to the present invention.

FIG. 4 shows the response curve of GOx/HRP-based array biosensor on the glucose determination according to the present invention.

FIG. 5 shows the response curve of uircase/HRP-based array biosensor for the determination of uric acid according to the present invention.

FIG. 6 shows the limits of detection, dynamic range, and reproducibility determined by sol-gel-derived array biosensor according to the present invention.

FIG. 7 shows experimental conditions and results of simultaneous detection of multi-analyte using the developed array biosensor according to the present invention.

FIG. 8 shows the 2-D plot of simultaneous determination on urea, creatinine, glucose, and uric acid using the array biosensor according to the present invention.

FIG. 9 shows the mixed effect of multi-analyte to the array biosensor according to the present invention.

FIG. 10 shows experimental conditions in the study of mixed effect of multi-analyte on the array biosensor according to the present invention.

FIG. 11 shows the reusability of the array biosensor according to the present invention.

FIGS. 12(A) to 12 (D) show the response of the array biosensor toward the standard addition of (A) urea, (B) creatinine, (C) glucose, and (D) uric acid into a serum sample.

### EXAMPLES

### Example 1

The Fabrication and Evaluation of a Sol-Gel Based Array Biosensor

1 Experimental Details

1.1 Reagents and Materials

Urease (EC 3.5.1.5, type IX, 54.3 units/mg-solid, from Jack beans), creatinine deiminase (CD, EC 3.5.4.21, 13 units/mg-solid, from Microorganism), glucose oxidase (GOx, EC 1.1.3.4, type X-S, 157.5 units/mg-solid, from Aspergillus niger), uricase (EC 1.7.3.3, 4.5 units/mg-solid, from Candida sp.), horseradish peroxidase (HRP, EC 1.11.1.7, type X, 291 units/mg-protein, from Horseradish), fluorescein isothiocyanate-dextran (FITC-dextran, MW 464,000), tetramethylrhodamine isothiocyanate-dextran (TRITC-dextran, MW 160,000), urea (99.5%), creatinine anhydrous (99%), uric acid (99%), and 1, 3-bis[tris(hydroxymethyl) methylamino]propane (BTP, 99%), were purchased from Sigma (St. Louis, Mo.). D-glucose (99%) was obtained from Icatayama Chemical (Osaka, Japan) and stock glucose solutions were allowed to mutarotate at room temperature for overnight before use. Tetramethyl orthosilicate (TMOS, 98%) was obtained from Tokyo Chemical Inc. (Tokyo, Japan). Polyvinylacetate (PVAc, MW 113,000) was purchased from Aldrich (Milwaukee, Wis.). Polyvinylalcohol (PVA, MW 205,000) was obtained from Fluka (Buchs, Switzerland). 10-acetyl-3,7-dihydrophenoxazine (Amplex red reagent, 95%) was obtained from Molecular Probes (Eugene, Oreg.). All other chemicals were of analytical grades and were used as received without further purification. A 50-well chambered coverslip (φ3 mm×1 mm, spacing 4.5 mm) was obtained from Grace Bio-Labs Inc. (Bend, Oreg., USA).

Urease (1090 U/mL) and CD (250 U/mL) were prepared by 5 mM BTP buffer at pH 7.5, while GOx/HRP (GOx: 650 U/mL, HRP: 950 U/mL) and Uricase/HRP (Uricase: 60 U/mL, HRP: 950 U/mL) were prepared by 5 mM BTP buffer at pH 6.0. Enzyme solutions were stored at 4° C. until used. The sol stock solution was prepared by adding 185 μL of TMOS and 45 μL of 1 mM HCl in a glass vial. The mixture was sonicated for 30 min at 20° C. to complete the acid-catalysis and to obtain a clear and homogeneous solution. The sol solution was stored at 4° C. and was utilized within 2 weeks. All aqueous solutions were prepared by bidistilled deionized water (bdH₂O) unless otherwise mentioned. The experiments were carried out in a clean room (Class 10000) and yellow lights were used to prevent the non-necessary photobleaching of dye molecules. The temperature was controlled at 19±2° C. and the relative humidity was in the range of 55 to 75%.

1.2 Preparation of the Array Biosensor Glass microscope slides (2.5 cm×7.5 cm) were cleaned with K₂Cr₂O₇/H₂SO₄ solution (0.84 g of K₂Cr₂O₇ was dissolved in 7 mL of H₂O and then 200 mL of concentrated H₂SO₄ was slowly added) for 2 h to remove the possible organic contaminants. After washing by copious amount of bdH₂O and drying under N₂ gas, the glass slides were dipped into PVAc solution (5% in dichloromethane) for 1 h to enhance the sol-gel attachment. The slides were taken out slowly from the container, purged with N₂, and stored under ambient conditions until used. A 50-well chambered coverslip was affixed on the PVAc-coated slide, and then placed on a thermoelectric cooler stage (TE cooler) for contact printing.

In preparing the urease and CD based biosensor, the sol-gel mixture was obtained by mixing 25 μL of fluorescent dye solution, which is containing FITC-dextran (1.25 mg/mL) and TRITC-dextran (2.5 mg/mL), 5 μL of glycerol, 7 μL of PVA (4% in H₂O), 10 μL of sol stock solution, and 5 μL of enzyme solution. In GOx/HRP and uricase/HRP biosensor, the sol-gel mixture was prepared by mixing 25 μL of 5 mM BTP buffer at pH 6.0, 5 μL of glycerol, 7 μL of PVA, 10 μL of sol stock solution, and 5 μL of enzyme solution. Immediately, 10 μL of the sol-gel mixture was printed on a coated glass slide automatically by a homemade robotic system. The 50 sol-gel spots (5×10), each with an individual well, were printed within 5 min and the spot size was around 800 μm (200 nL). Subsequently, the array was allowed for gelation for 15 min under ambient conditions. Ten μL of BTP buffer was subsequently loaded into the wells to stabilize the sol-gel for 1 h prior to being used. By a means of contact printing, the volume of sol-gel spot could be reduced to 200 nL, which is significantly lower than that in direct deposition method. Therefore, the developed technique was suitable for the analytical applications of the proteins that available only in a trace amount.

1.3 Apparatus

1.3.1 Pin printing system

An automated homemade screen-printing and fluorescence detection systems for the fabrication and detection of sol-gel-derived array biosensor were developed. The constructed array printer contains three motorized stages, which are responsible for X-Y-Z translations. The stepping and positioning resolutions were of 1.25 μm and 10 μm, respectively. To simplify and compact the whole biosensor array fabrication and detection systems, the X and Y translation stages of the printer were also shared to the fluorescence detection system.

In addition, a LabVIEW program (National Instruments, TX, USA) was designed to offer a user-friendly interface for controlling the translation stages and the automated printing of the sol-gel array in the desired pattern. However, the LabVIEW program is not described in detail herein since it is not a feature of the present invention.

1.3.2 Fluorescence Detection

The fluorescence microscope (ECLIPSE E600, Nikon Corp., Tokyo, Japan) equipped with a blue-enhanced silicon detector with a 100 mm² (Edmund Industrial Optics Inc., Barrington, N.J., USA) sensing area was used for the fluorescence detection. The intensity of the light source (100 W mercury lamp) was decreased to 1/32 of the original value using neutral density filters in order to prevent the photobleaching of the dye molecules. Two filter blocks, G-2A (excitation (EX) 510-560 nm, dichroic mirror (DM) 575 nm, barrier filter (BA) 590 nm, Nikon) and B-2A (EX 450-490 nm, DM 505 nm, BA 520 nm, Nikon), were used for the detection of TRITC-dextran and FITC-dextran, respectively. An optical chopper with 5/6 slot blade (SR540, Stanford Research Systems, Sunnyvale, Calif., USA) was set at the frequency of 392 Hz to minimize the electronic interference. The signal obtained from the silicon detector was integrated with the chopper reference by a single-board lock-in amplifier (FEMTO Messtechnik GmbH, Berlin, Germany). By using this technique, we can not only enhance the sensitivity of the biosensor but also provide the potentiality on miniaturization. Finally, the data was acquired by the data acquisition card (DAQ card) (PCI-1200, National Instruments, TX, USA). Fluorescence detection was carried out automatically by scanning over the sol-gel spots by the developed robotic system programmed by the LabVIEW™ software.

1.3.3 Total Immersion System

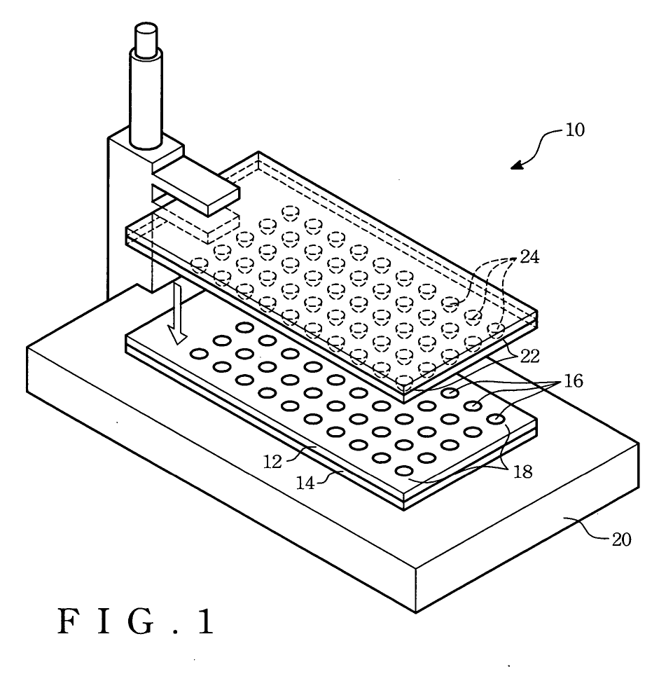

The development of sol-gel based biosensor array provides an opportunity to detect multi-analyte simultaneously since each sol-gel element is isolated in an independent well. This design allowed the array biosensor to proceed different reactions simultaneously. However, 5 min is usually required to complete the manual loading of different solutions into 50 wells, which causes different reaction times among sensing elements throughout the entire array and induces unreliable results. This issue becomes an important consideration when a relatively fast enzymatic reaction was observed. Referring to FIG. 1, a total immersion device 10 was developed to solve this potential drawback of the array biosensor. The 50-well coverslip 12 was fixed on a clean and non-coated glass slide 14 to allow the loading of different solutions 16, i.e. the solution array 18. Then, the solution array 18 was placed on a two-axis tilt platform 20 which could adjust the slide 14 horizontal. The sol-gel array 22 was mounted inversely on a precision translation stage, which permitted the immersion of all the sol-gel spots 24 into the corresponding solutions 16 of the solution array 18. By using this device, we can ensure that the sol-gel elements have the same reaction time with the variations smaller than 5 sec.

1.4 Principles

For determinations of renal-related metabolites, two major categories of enzymes were used. Urease and CD belong to the hydrolase while GOx, uircase, and HRP are types of oxidase. Hence, two different principles based on the pH change and the reduction of the Amplex red reagent were designed for different types of biosensors. In the enzymatic hydrolysis of urea and creatinine, hydroxide ions are produced and can be readily detected via an immobilized pH-sensitive fluorescent indicator, FITC-dextran, giving an increase in fluorescent intensity at a maximum wavelength of 520 nm. Another pH-insensitive dye probe, TRITC-dextran, was also encapsulated in silica to calibrate possible error arising from different printed sizes of sol-gel spots and defocusing problems. The maximum wavelength of emission fluorescence of TRITC-dextran is at 570 nm. Thus, the fluorescence intensity of FITC over TRITC (FT ratio) was determined using the developed fluorescence detection system and was used to present the response of biosensors with respect to various concentrations of target. Schemes 1 and 2 show the hydrolysis reactions of urea and creatinine catalyzed by urease and CD, respectively.

Another strategy involving two enzymatic reactions was designed for glucose and uric acid biosensors. As shown in Schemes 3 and 4, β-D-glucose (or uric acid) was first oxidized by GOx (or uricase) with a generation of H₂O₂ and gluconolactone (or allantoin). Subsequently, hydrogen peroxide was consumed by HRP to give a strong fluorescence at a maximum wavelength of 590 nm as a result of reduction of Amplex red to resorufin. Therefore, in this study, GOx (or uricase) and HRP were co-immobilized in sol-gel matrix for glucose and uric acid determination, respectively.

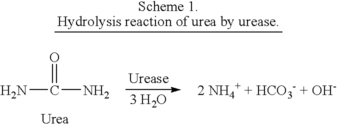

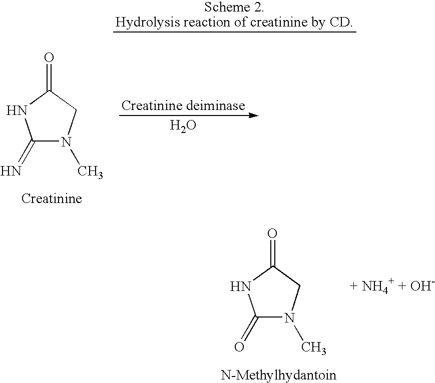

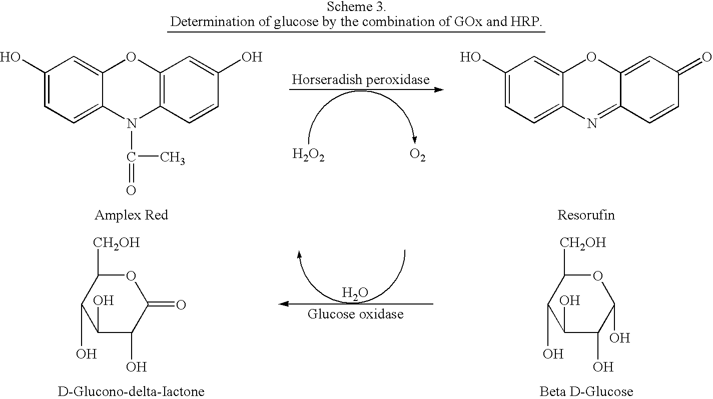

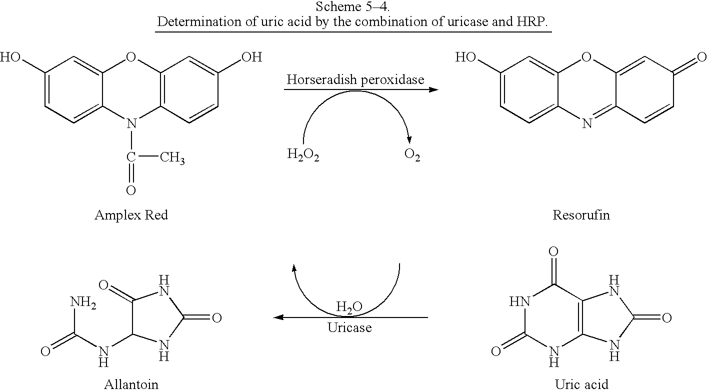

### Example 2

Results and Discussion

2 Experimental Details

2.1 Urea Determination

Urea constitutes the major excretory product of protein metabolism and thus is predominant non-protein nitrogenous substance in the blood, comprising more than 75% of the non-protein nitrogen eventually excreted. BUN (Blood Urea Nitrogen) has now been accepted as a general marker for kidney function. In the present invention, a pH-sensitive fluorescent dye, FITC-dextran, was co-encapsulated with urease in sol-gel network to reply a pH change of the system. Since hydroxide ions were generated during the urea hydrolysis, FITC would give an increase in fluorescent intensity proportional to the urea concentration.

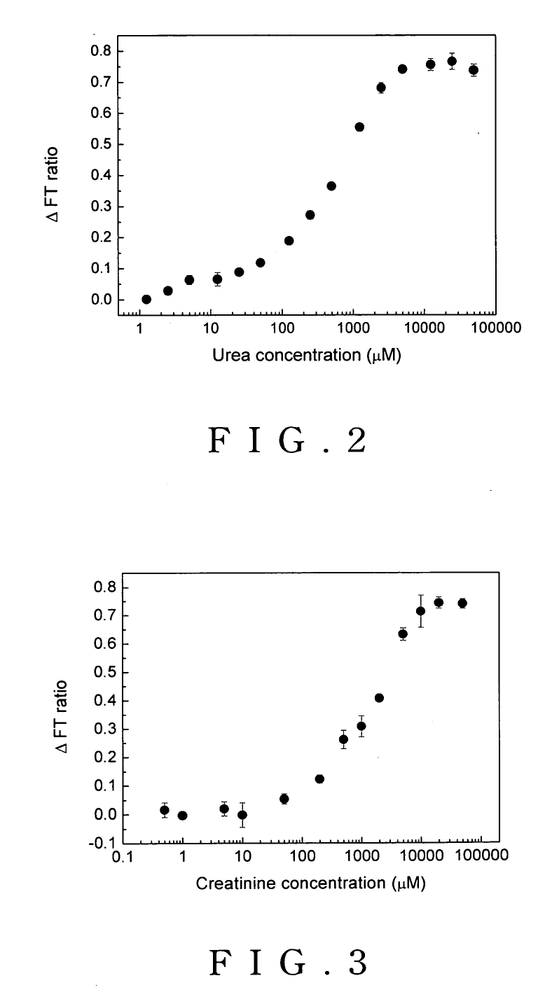

FIG. 2 shows the response curve of the array biosensor in the presence of various concentrations of urea ranging from 1.25 μM to 50 mM. The urea was prepared in 1 mM BTP buffer at an initial pH 6.0 and was allowed to react with urease-entrapped sol-gel for 10 min. A sigmoidal curve (S-shape) as a function of urea concentration with a dynamic range of 3 orders of magnitude was observed, indicating that a possible kinetic restriction of the enzymatic reaction may be involved. The delta FT ratio (ΔFT ratio), normalized to the buffer solution, increased rapidly with increasing amounts of urea and leveled off at the concentration of 10 mM. A limit of detection, defined by the t-test with p<0.05, of 2.5 μM was obtained. The sensitivity of the developed array biosensor is sufficiently low for the determination of urea in serum, which the normal range of BUN is 2.5-8 mM.

The reproducibility of the biosensor was also investigated in this application. Since the inflection point (middle of the S-curve) usually exhibits large variations, the concentration located in the inflection point (500 μM) is used to treat 50 sol-gel spots for 10 min for the examination of the reproducibility of the array biosensor. The relative standard deviation (RSD) of 4.8% (n=45) was observed through the entire array, depicting that all the sol-gel elements can be maintained in a good manner of reproducibility under the designed analytical procedure and has excellent sensitivity for the determination of urea.

2.2 Creatinine Determination

Creatinine is a metabolic byproduct of muscle metabolism and approximately 1-2% of muscle creatine is converted to creatinine daily. Increasing use is being made of plasma creatinine levels alone for the assessment of renal function and it has been shown that plasma creatinine is more sensitive than creatinine clearance in detection change in glomerular function. In this study, creatinine determination was accomplished using the CD (creatinine deiminase)-based sol-gel-derived array biosensor.

As shown in FIG. 3, a large excess of substrate concentration causes the first-order enzymatic reaction shift to a zero-order reaction, giving an independent reaction rate versus to the further increase of the substrate concentration. Therefore, a similar sigmoidal curve as in the urea calibration dependency was also observed in creatinine measurement. A dynamic range of 3 orders of magnitude and a detection limit of 50 μM (t-test, p<0.05) were obtained. The normal ranges for creatinine in serum are 40-130 μM. However, in patients with kidney dysfunction, creatinine levels can rise to a concentration higher than 1 mM. Therefore, although the detection limit of the array biosensor is located in the normal range of serum creatinine, the system still can be used in the case of acute or chronic renal failure. Moreover, it is worth pointing out that the developed system is the first sol-gel-based biosensor for the creatinine determination. This means that the versatile and easy-used sol-gel technique was also suitable to immobilize CD as well as to fabricate a biosensor for the measurement of creatinine.

2.3 Glucose Determination

Glucose biosensor is one of the most well known biosensors now developed and is commercially available due to its importance for patients suffered from diabetes mellitus. Diabetes is also the leading cause of end-edge renal disease (ESRD) (i.e., kidney failure requiring dialysis or kidney transplantation). The incidence of ESRD attributed to diabetes mellitus (ESRD-DM) treatment is increasing among American Indians/Alaska Natives.

In the present invention, GOx and HRP were co-immobilized in sol-gel to proceed a two-step reaction which converted H₂O₂ released from glucose oxidation to O₂ associated with the reduction of Amplex red reagents. A working solution containing 50 mM BTP buffer at pH 7.5, 5 μM Amplex red, 62.5 μg/mL (0.13 μM) FITC-dextran, and various concentration of glucose ranging from 8 μM to 4 mM were tested by the developed array biosensor. Here, FITC-dextran was used as a reference marker to calibrate the concentrated effect of Amplex red as a result of liquid evaporation during fluorescence detection. Because only a small volume of sample (15 μL) was required for analyses, the increase in Amplex red concentration when solvent evaporated may be important. Therefore, the fluorescence intensity of Amplex red over the FITC (AF ratio) was plotted in response to various glucose concentrations.

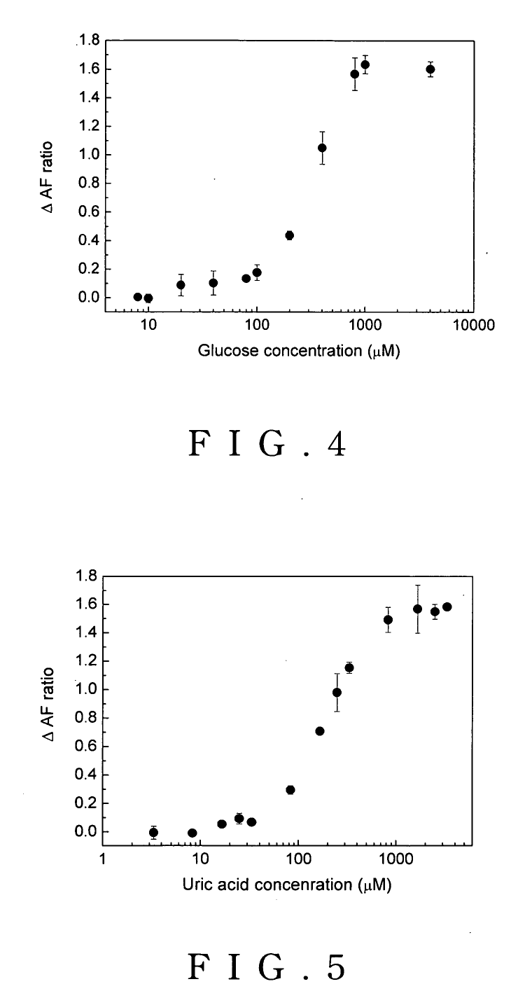

FIG. 4 shows the calibration curve of GOx/HRP-coimmobilized array biosensor on the glucose determination expressing by a difference of AF ratio (ΔAF ratio). The ΔAF ratio was obtained by subtracting the AF ratio in the presence of glucose from that when glucose was absent. Basically, A AF ratio increased with the increase in glucose concentration and reached a plateau at an approximate concentration of 1 mM. The dynamic range of 10 to 1000 μM with a detection limit (t-test, p<0.01) of 80 μM could be obtained. This range was significantly lower than the normal range of glucose in serum (3.5-5.8 mM). It reveals that a dilution process of serum sample may be required when applying this biosensor for glucose analysis.

2.4 Uric Acid Determination

The participation of uricase in the final step of purine degradation caused the release of uric acid, an important marker for disorders associated with purine metabolism, most notably gout and hyperureicaemia. Moreover, since uric acid is only slightly soluble in water, it may also precipitate and contribute to the formation of kidney stones.

The detection of uric acid was conducted in a similar manner as in the glucose measurements except the encapsulation of uricase/HRP in sol-gel material instead of using GOx/HRP. FIG. 5 demonstrates the response of the array biosensor as a function of concentrations of uric acid prepared in 50 mM BTP buffer at pH 7.5. A sigmoidal curve with a dynamic range of 2 orders of magnitude could be observed. The limit of detection was 25 μM (t-test, p<0.05), which was lower enough to fulfill the normal range of uric acid in serum (0.09-0.42 mM).

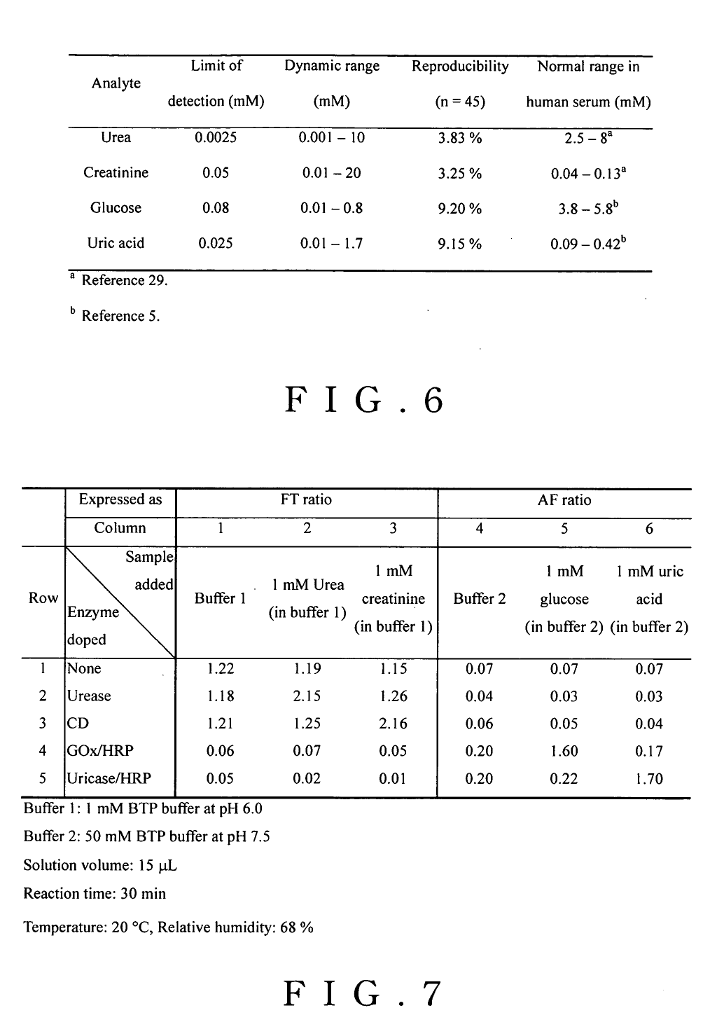

The normal range and clinical significances of urea, creatinine, glucose, and uric acid in human serum as well as the analytical performance of the developed array biosensor in response to these substrates were summarized in FIG. 6. Results show that the sol-gel based array biosensors developed in this work are able to determine urea, creatinine, glucose and uric acid and exhibit good analytical performances in terms of sensitivity, reproducibility and dynamic range. Although the detection limit of this system for creatinine measurement was located in the normal concentration range in serum, the biosensor still could be useful in analyzing the kidney dysfunction (serum creatinine >1 mM).

2.5 Simultaneous Determination of Multi-Analyte

One of the major advantages of an array-based biosensor is the capability of simultaneous determination of multi-analyte when different sol-gel spots were immobilized with different enzymes. Since each sol-gel spot has its own reaction chamber in the array biosensor, it provides another advantage of simultaneous detection of multi-sample by simply adding different samples into different reaction chambers. This is contrast to the conventional microarray, which is spotted with thousands of probes each recognized different analytes but usually only one sample can be determined at a time. Biosensors, different from the microarray, have typically less analytes (<hundreds) but large numbers of samples especially when clinical (or environmental) applications are aimed. Therefore, highly parallel and rapid analyses become one of the major concerns in the development of biosensors. That is also the reason why some biosensors were specially designed for continues monitoring or repeated use. To understand the potentially for the simultaneous determination of multi-analyte and multi-sample, a sol-gel-derived array biosensor encapsulated with urease, CD, GOx/HRP, and uricase/HRP in different rows was fabricated.

As shown in FIG. 7, different enzymes (or without enzyme) were printed in different rows, while samples were added in different columns. FIG. 8 shows the 2-D plot of simultaneous detection of urea, creatinine, glucose, and uric acid using array biosensor with different enzymes. The first three columns (column 1-3) for the detections of buffer 1 (1 mM BTP at pH 6.0), urea, and creatinine were expressed by FT ratio while another three columns (column 4-6) for the measurements of buffer 2 (50 mM BTP at pH 7.5), glucose, and uric acid were described by AF ratio. This discrepancy is due to the different principles in pH- and Amplex red-based biosensors. A similar FT ratio (RSD=1.7%, n=3) in response to buffer 1 was observed in non-doped, urease- and CD-entrapped spots, depicting that the co-immobilization of FITC-dextran with different enzymes in sol-gel matrix did not affect the sensitivity of FITC to pH values (column 1). Results also showed that the developed pH-based biosensor exhibited a good specificity to urea and creatinine detections, in which 1 mM substrate was recognized and only recognized by its corresponding enzyme (column 2 and 3).

Amplex red is a very stable reagent and can be converted to resorufin only when HRP and H₂O₂ are coexisted. This phenomenon could be revealed by a low background fluorescence (AF ratio=0.2, defined as the fluorescence intensity of amplex red over that of FITC) in glucose and uric acid biosensors when only working solutions (no substrates) were treated (column 4). Similar AF ratios were found when the enzyme was treated with an incongruent substrate e.g. GOx with uric acid (column 5 and 6), depicting that no obvious cross-talk effect was existed between glucose and uric acid. When the enzyme and substrate pair was matched, a good specificity for the detection of glucose and uric acid was then observed (column 5 and 6). Overall, results reveal that the sol-gel-derived array biosensor developed in this study presents a good specificity and is applicable for the simultaneous detection of multi-analyte including urea, creatinine, glucose and uric acid.

2.6 Mixed Effect of Multi-Analyte

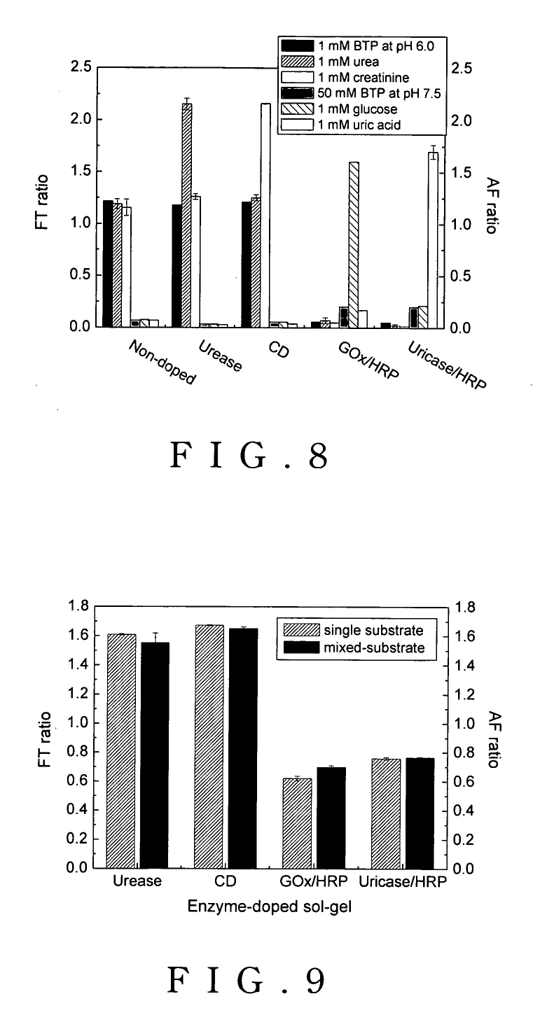

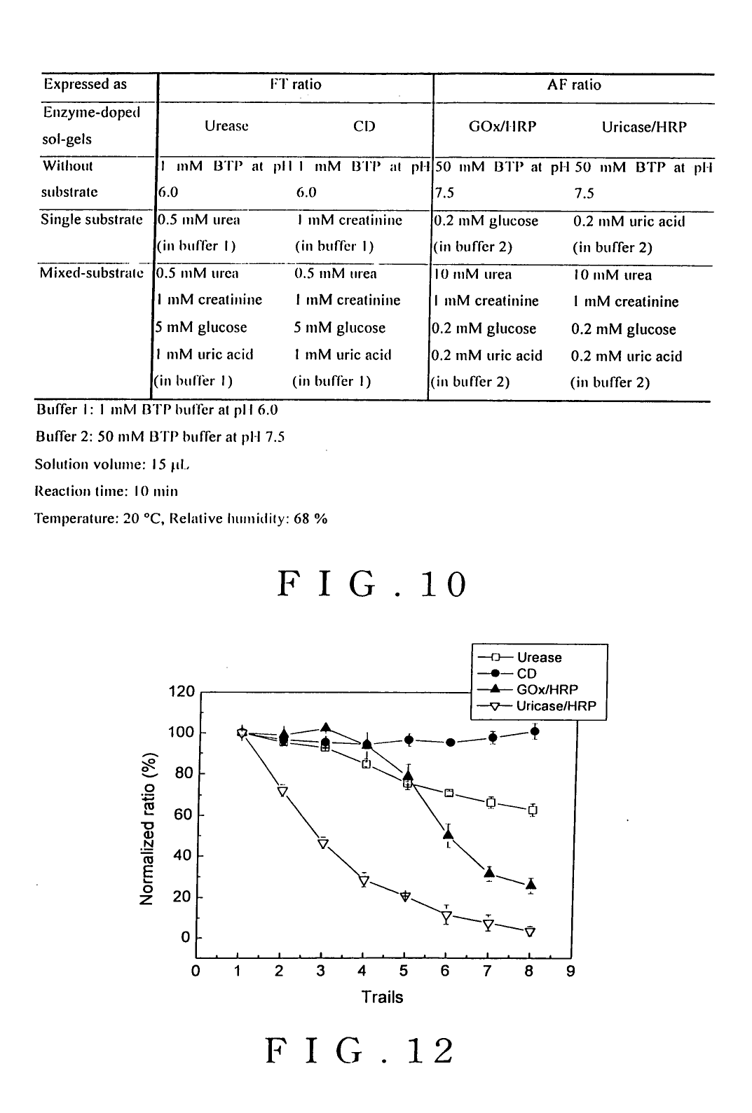

Although the array biosensor with multi-enzymes showed obvious responses when corresponding substrates were added, antagonistic or synergistic effects might occur when different analytes were mixed. Therefore, the mixed effect of multi-analyte on the performance of array biosensor was also studied. FIG. 9 shows the responses of biosensor when single substrate or mixed-analytes are amended. The experimental condition for the examination of mixed effect of multi-analyte on the biosensor is shown in FIG. 10. When urea or creatinine were determined, high levels of glucose and uric acid up to 5 mM and 1 mM, respectively, were used for the mixed-analyte experiment. Similarly when glucose and uric acid were aimed, high levels of urea and creatinine up to 10 mM and 1 mM, respectively, were used for the preparation of mixed-analyte. Generally, no obvious cross-interaction of the mixed analytes to the array biosensor was found. Although the glucose biosensor showed a slight increase in response when registered with mixed substrates, the relative deviation was at 7.6%, which is still in the range of analytical error. This means that the array biosensor encapsulated with different enzymes exhibits a good specificity even in a mixed-analyte solution. This also implies the possibility of multi-analyte detection in complex mediums via the fabricated biosensors.

2.7 Reusability of the Array Biosensor

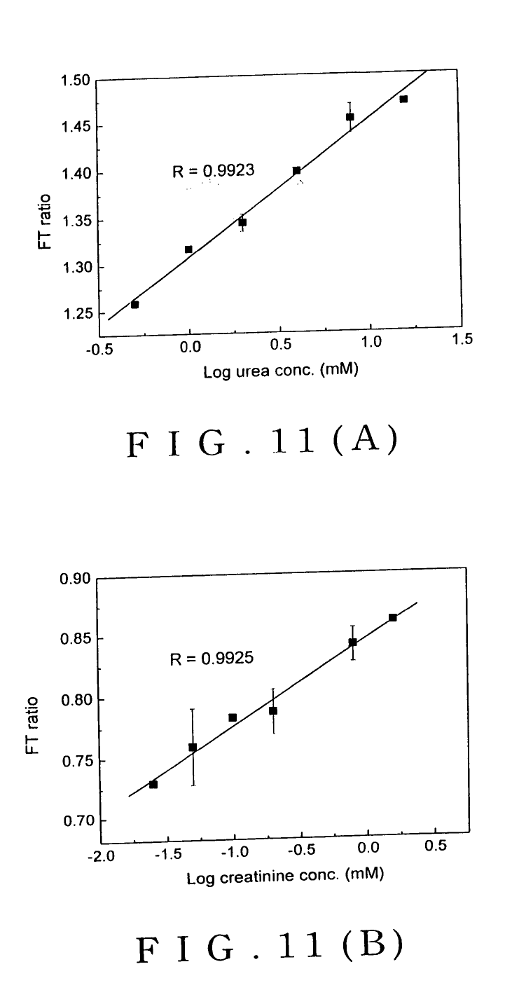

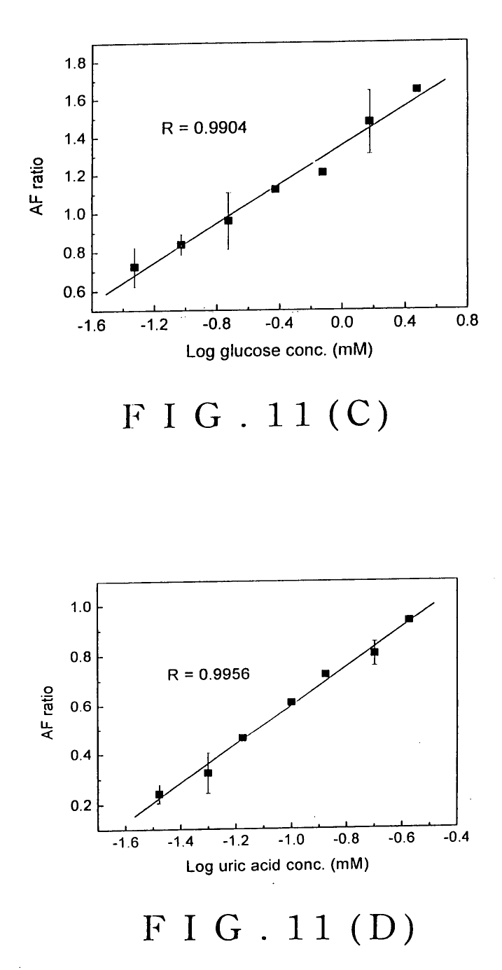

The reusability of array biosensors was investigated in this study to evaluate the superiority of the developed biosensor. The reusability experiment was carried out by repeatedly treating the enzyme-encapsulated sol-gel spot with substrate for 8 times. As shown in FIG. 11, urea, creatinine, and glucose biosensor exhibits a good reusability in which the response of biosensor can be maintained higher than 75% of the original value after 5 times use. Uric acid biosensor showed relatively poor reusability and the response decrease obviously along with the repeated trials. This decrease may be attributed to the block of the active sites as a result of releasing of reaction products.

2.8 Standard Addition of a Serum Sample

In order to evaluate the applicability of the system to real sample analysis, serum sample spiked with different analytes at various concentrations were tested using the array biosensor. FIG. 12 shows semi-logarithm plot of the response of the array biosensor in sensitive to a serum sample (Foetal Calf Serum) with the addition of different substrates. The response curves in serum sample have different slopes when compared to the calibration curves obtained in buffer solution, which represents that the matrix effect could significant alter the biosensor responses. The recovery for urea, creatinine and glucose at 1 mM, and uric acid at 0.1 mM were 140, 83, 72, and 114%, respectively. Although the poor recovery was obtained, good linear relationships could be observed for all tested substrates with correlation coefficients of larger than 0.99. This implies that the array biosensor might still be useful for analyzing metabolites in serum samples since the real concentrations could be calculated by fitting the results of standard addition experiment.

Although the preferred embodiment of this invention has been disclosed for illustrative purpose, those skilled in the art will appreciate that various modifications, additions and substitutions are possible, without departing from the scope and spirit of the invention as described in the accompanying claims.

1. A method of detecting the concentration of an analyte in a plurality of biological samples, comprising:
a) providing:
i) a detecting device having a plurality of wells formed thereon, wherein a composition comprising a catalyst, a first fluorescent dye and a second fluorescent dye is encapsulated in sol-gel within said wells;
ii) a substrate having an array of said biological samples prepared thereon;
b) contacting said detecting device with said substrate such that said analyte in said biological samples reacts with said composition in said wells causing a change in spectroscopic property, wherein the change in spectroscopic property is calibrated by said second fluorescent dye for normalization; and
c) comparing the calibrated spectroscopic property to a standard curve to determine the concentration of said analyte in said biological samples.

2. The method of claim 1, wherein said sol-gel comprises tetramethylorthosilicate.

3. The method of claim 1, wherein said biological samples comprise human blood samples.

4. The method of claim 1, wherein said catalyst comprises an enzyme.

5. The method of claim 1, wherein said first fluorescent dye comprises fluorescein isothiocyanate.

6. The method of claim 1, wherein said second fluorescent dye comprises tetramethylrhodamine isothiocyanate.

7. A method of detecting the concentration of multiple analytes in a biological sample, comprising:
a) providing:
i) a detecting device having a plurality of wells formed thereon, each of said well having a respective composition loaded therein, wherein said composition comprising a catalyst, a first fluorescent dye and a second fluorescent dye is encapsulated in sol-gel within said well;
ii) a substrate having an array of said biological sample prepared thereon;
b) contacting said detecting device with said substrate such that said analytes in said biological sample react with the composition within each of said well causing a change in spectroscopic property, wherein the change in spectroscopic property is calibrated by said second fluorescent dye for normalization; and
c) comparing the calibrated spectroscopic property to a standard curve to determine the concentration of said analytes in said biological sample.

8. The method of claim 7, wherein said sol-gel comprises tetramethylorthosilicate.

9. The method of claim 7, wherein said biological sample comprises human blood samples.

10. The method of claim 7, wherein said catalyst comprises an enzyme.

11. The method of claim 7, wherein said first fluorescent dye comprises fluorescein isothiocyanate.

12. The method of claim 7, wherein said second fluorescent dye comprises tetramethylrhodamine isothiocyanate.

13. A method of detecting the concentration of an analyte in a plurality of biological samples, comprising:
a) providing:
j) a detecting device having a plurality of wells formed thereon, wherein a composition comprising a catalyst is encapsulated in sol-gel within said wells;
ii) a substrate having an array of a plurality of sample solutions prepared thereon, wherein each of said sample solution comprises one of said biological samples, a first fluorescent dye and a second fluorescent dye;
b) contacting said detecting device with said substrate such that said analyte in said biological samples reacts with said composition in said wells causing a change in spectroscopic property, wherein the change in spectroscopic property is calibrated by said second fluorescent dye for normalization; and
c) comparing the calibrated spectroscopic property to a standard curve to determine the concentration of said analyte in said biological samples.

14. The method of claim 13, wherein said sol-gel comprises tetramethylorthosilicate.

15. The method of claim 13, wherein said biological samples comprise human blood samples.

16. The method of claim 13, wherein said catalyst comprises an enzyme.

17. The method of claim 13, wherein said first fluorescent dye comprises Amplex red.

18. The method of claim 13, wherein said second fluorescent dye comprises fluorescein isothiocyanate.

19. A method of detecting the concentration of multiple analytes in a biological sample, comprising:
a) providing:
j) a detecting device having a plurality of wells formed thereon, each of said well having a respective composition loaded therein, wherein said composition comprising a respective catalyst is encapsulated in sol-gel within said well;
ii) a substrate having an array of a sample solution prepared thereon, wherein said sample solution comprises said biological sample, a first fluorescent dye and a second fluorescent dye;
b) contacting said detecting device with said substrate such that said analytes in said biological sample react with the composition within each of said well causing a change in spectroscopic property, wherein the change in spectroscopic property is calibrated by said second fluorescent dye for normalization; and
c) comparing the calibrated spectroscopic property to a standard curve to determine the concentration of said analytes in said biological sample.

20. The method of claim 19, wherein said sol-gel comprises tetramethylorthosilicate.

21. The method of claim 19, wherein said biological sample comprises human blood samples.

22. The method of claim 19, wherein said catalyst comprises an enzyme.

23. The method of claim 19, wherein said first fluorescent dye comprises Amplex red.

24. The method of claim 19, wherein said second fluorescent dye comprises fluorescein isothiocyanate.
